(19) United States
(12) Patent Application Publication
(10) Pub. No.: US 2023/0295263 A1
(43) Pub. Date: Sep. 21, 2023

(54) SOLUBLE THY-1 COMPOSITIONS AND USE THEREOF TO TREAT OR REVERSE FIBROSIS

(71) Applicants: The University of North Carolina at Chapel Hill, Chapel Hill, NC (US); The Regents of the University of California, Oakland, CA (US)

(72) Inventors: James Hagood, Chapel Hill, NC (US); Simon Wong, La Jolla, CA (US); Ronit Fraiman, Chapel Hill, NC (US)

(21) Appl. No.: 18/303,796

(22) Filed: Apr. 20, 2023

Related U.S. Application Data
(62) Division of application No. 17/514,954, filed on Oct. 29, 2021.
(60) Provisional application No. 63/107,102, filed on Oct. 29, 2020.

(51) Int. Cl. C07K 14/705; A61P 11/00; C12N 15/63

(52) U.S. Cl. CPC C07K 14/70503; A61P 11/00; C12N 15/63; A61K 38/00

(57) ABSTRACT

The invention relates to a soluble Thy-1 polypeptide or a functional fragment thereof and pharmaceutical compositions including the soluble Thy-1 polypeptide or a functional fragment thereof. The invention also relates to the use of the soluble Thy-1 polypeptide or a functional fragment thereof for delivery to subjects with tissue fibrosis for treating, inhibiting, and/or reversing tissue fibrosis in the subject.

### STATEMENT OF PRIORITY

This patent application is a divisional of and claims priority to U.S. patent application Ser. No. 17/514,954, filed on Oct. 29, 2021, which claims the benefit under 35 U.S.C. § 119(e), of U.S. Provisional Application No. 63/107,102, filed on Oct. 29, 2020, the entire contents of each of which are incorporated by reference herein.

### STATEMENT OF GOVERNMENT SUPPORT

This invention was made with government support under Grant Numbers HL082818 and HL111169-01A1 awarded by the National Institutes of Health. The government has certain rights in the invention.

### STATEMENT REGARDING ELECTRONIC FILING OF A SEQUENCE LISTING

A Sequence Listing in XML format, submitted under 37 C.F.R. § 1.821, entitled 5470-918DV_ST26.xml, 14,311 bytes in size, generated on Apr. 18, 2023 is filed electronically herewith. This Sequence Listing is hereby incorporated herein by reference into the specification for its disclosure.

### FIELD OF THE INVENTION

This invention relates to a soluble Thy-1 polypeptide or a functional fragment thereof and pharmaceutical compositions including the soluble Thy-1 polypeptide or a functional fragment thereof. The invention also relates to the use of the soluble Thy-1 polypeptide or a functional fragment thereof for delivery to subjects with tissue fibrosis for treating, inhibiting, and/or reversing tissue fibrosis in the subject.

### BACKGROUND OF THE INVENTION

Progressive pulmonary fibrosis occurs worldwide and is nearly uniformly fatal. Current FDA-approved medication has only decelerated the ongoing tissue remodeling, rather than halting or reversing it. There are clearly significant gaps in understanding impaired tissue remodeling leading to progressive fibrosis, characterized by persistent or aberrant fibroblast activation and excessive deposition of extracellular matrix (ECM) in various tissues including the lungs. It is clear that fibrotic tissue remodeling can progress via multiple feed-forward amplification loops if the effector cells, myofibroblasts, acquire certain fibrotic phenotype characteristics, such as apoptosis resistance, restricted dedifferentiation and compromised autophagy. At the same time, excessive and persistent abnormal ECM promotes further activation of fibroblasts and differentiation to myofibroblasts. Therefore, interruption of ongoing fibroblast activation and elimination or inactivation of myofibroblasts are critical for fibrosis resolution.

Targeting of αv integrin has identified a core molecular pathway that regulates organ fibrosis. αv integrins are expressed in collagen-positive fibroblasts and are upregulated and activated in αSMA-positive myofibroblasts, contractile cells capable of exerting force on tethered ligands, resulting in integrin-dependent activation of latent TGF-β. Specifically, αv integrins interact with an arginine-glycine-aspartic acid (RGD) motif present in the latency-associated peptide, which maintains TGF-β in an inactive state in the extracellular matrix. Active TGF-β can be released from the latency-associated peptide following αv integrin binding and myofibroblast contraction. In mice, genetic depletion of the αv integrin subunit in myofibroblasts using Pdgfrb-Cre system was protective against organ fibrosis, including lungs. αv integrin blockade also reduces TGF-β activation in primary human skeletal muscle and cardiac PDGFRβ+ cells. Clearly, these studies have indicated that αv integrins are highly expressed and targetable on myofibroblasts, thus representing a potential novel therapeutic option in the treatment of fibrotic diseases.

Thy-1, a GPI-anchored glycoprotein, contains an integrin-binding RGD-like motif (RLD) and regulates the phenotype of fibroblasts. Thy-1 is highly expressed on normal fibroblasts, but absent in activated fibroblasts and myofibroblasts in idiopathic pulmonary fibrosis (IPF) fibroblastic foci (FF), and decreases with aging. In vitro, Thy-1 expression determines the ability of lung fibroblasts to activate in response to fibrogenic stimuli. Thy-1 (−) lung fibroblasts are hyper-proliferative, differentially express growth factor receptors, display altered cytokine signaling, showing enhanced myofibroblastic differentiation, apoptosis resistance, and enhanced activation of latent TGF-β. Notably, interactions of soluble Thy-1 with αvβ5 integrin inhibited lung fibroblast contraction-induced latent TGF-β1 activation and myofibroblast differentiation. Moreover, Thy-1 physically couples to inactive αvβ3 integrin, altering baseline integrin avidity to ECM ligands and facilitating preadhesion clustering of integrins and membrane rafts via Thy-1's glycophosphatidylinositol tether. Disruption of Thy-1-αvβ3 coupling altered recruitment of Src family kinases to adhesion complexes and impaired mechanosensitive, force-induced Rho signaling, and rigidity sensing, sufficient to induce myofibroblast differentiations in normal, physiologically soft ECMs. These in vitro findings highlight the role of Thy-1 in αv integrin-dependent TGF-β1 activation and downstream profibrotic signaling in fibroblasts, however, its therapeutic relevance in pathological fibrosis has not been determined.

### SUMMARY OF THE INVENTION

This invention is based on the finding that the use of a soluble Thy-1 polypeptide or a functional fragment thereof for delivery to subjects with tissue fibrosis is effective for Thy-1 activity, thereby treating and even reversing the tissue fibrosis. Thus, one aspect of the invention relates to the use of a soluble Thy-1 polypeptide or a functional fragment thereof or a fusion protein comprising a soluble Thy-1 polypeptide or a functional fragment thereof for delivery to subjects with tissue fibrosis for treating, inhibiting, and/or reversing tissue fibrosis in the subject. Another aspect of the invention is reversing profibrotic activation of a fibroblast using a soluble Thy-1 polypeptide or a functional fragment thereof or a fusion protein comprising a soluble Thy-1 polypeptide or a functional fragment thereof.

In some embodiments, the fibrosis is a self-resolving fibrosis. In other embodiments, the fibrosis is a non-resolving fibrosis. The fibrosis can be in any tissue or organ. In one embodiment, the fibrosis is lung fibrosis, optionally the lung fibrosis is idiopathic lung fibrosis. In other embodiments, the fibrosis is kidney, liver, or heart fibrosis. In other embodiments, the fibrosis is in arthritic tissue.

Another aspect of the invention relates to the use of a soluble Thy-1 polypeptide or a functional fragment thereof for inhibiting alveolar septal thickening in a subject.

Another aspect of the invention relates to treating or preventing a disorder responsive to inhibition or reversal of tissue fibrosis in a subject by delivering to the subject a therapeutically or prophylactically effective amount of a soluble Thy-1 polypeptide or a functional fragment thereof or a fusion protein comprising a soluble Thy-1 polypeptide or a functional fragment thereof.

In some embodiments, the soluble Thy-1 polypeptide or a functional fragment thereof is delivered by inhalation. In other embodiments, the soluble Thy-1 polypeptide or a functional fragment thereof is delivered systemically, such as intravenously.

In some embodiments, the fusion protein comprising a soluble Thy-1 polypeptide or a functional fragment thereof is a fusion with an immunoglobulin Fc region. In other embodiments, the fusion protein comprising a soluble Thy-1 polypeptide or a functional fragment thereof does not comprise an immunoglobulin Fc region. In some embodiments, the functional fragment of Thy-1 is a deletion of a glycosylphosphatidyl inositol attachment signal.

In some embodiments, Thy-1 is human Thy-1. In some embodiments, the subjects are human subjects.

Another aspect of the invention relates to a fusion protein comprising a soluble Thy-1 polypeptide or a functional fragment thereof that does not comprise an immunoglobulin Fc region. In some embodiments, the functional fragment of Thy-1 is a deletion of a glycosylphosphatidyl inositol attachment signal. In some embodiments, the Thy-1 is human Thy-1.

Another aspect of the invention relates to a pharmaceutical composition comprising a soluble Thy-1 polypeptide or a functional fragment thereof or a fusion protein comprising a soluble Thy-1 polypeptide or a functional fragment thereof and a pharmaceutically acceptable carrier. In some embodiments, the soluble Thy-1 polypeptide or a functional fragment thereof is a fusion with an immunoglobulin Fc region. In other embodiments, the soluble Thy-1 polypeptide or a functional fragment thereof does not comprise an immunoglobulin Fc region. In some embodiments, the functional fragment of Thy-1 is a deletion of a glycosylphosphatidyl inositol attachment signal. In some embodiments, the Thy-1 is human Thy-1.

Another aspect of the invention relates to a kit with a soluble Thy-1 polypeptide or a functional fragment thereof or a fusion protein comprising a soluble Thy-1 polypeptide or a functional fragment thereof and a pharmaceutically acceptable carrier. In some embodiments, the soluble Thy-1 polypeptide or a functional fragment thereof is a fusion with an immunoglobulin Fc region. In other embodiments, the soluble Thy-1 polypeptide or a functional fragment thereof does not comprise an immunoglobulin Fc region. In some embodiments, the functional fragment of Thy-1 is a deletion of a glycosylphosphatidyl inositol attachment signal. In some embodiments, the Thy-1 is human Thy-1.

Another aspect of the invention relates to a dosage delivery device including the pharmaceutical composition, optionally wherein the dosage delivery device is an inhaler.

### BRIEF DESCRIPTION OF THE DRAWINGS

FIGS. 1A-1D show the transient Thy1 loss in GFP (+) fibroblasts in a self-resolving model of lung fibrosis induced with single dose bleomycin (Bleo). FIG. 1A is a series of immunofluorescence (IF) images of lungs with Col1α1-GFP, Thy1, and nuclei overlaid for the entire viewing field and include a magnified view of Thy1 below. Scale bar: 100 μm. FIG. 1B is the experimental scheme, showing that Adult Col1α1-GFP mice (n=5-7/group) were given Bleo (4 U/kg in 100 μL saline) by orotracheal intubation/MicroSprayer at day zero. Lung samples were collected at 28 or 56 days after Bleo instillation. FIG. 1C is a series of bar graphs of the positively stained area quantified as a total density using ImageJ software (NIH). FIG. 1D is a series of bar graphs of mRNA expression of Thy1 and fibrogenic genes (Col1a1, Col3a1) determined by qPCR. Results are presented as Mean±SEM. Statistical analysis was performed using one-way ANOVA; **P<0.01.

FIGS. 2A-2D show persistent Thy-1 loss associated with silenced expression in GFP (+) fibroblasts in the non-resolving model of lung fibrosis induced with repeated bleomycin (Bleo) dosing. FIG. 2A is a series of immunofluorescence (IF) images of lungs with Col1α1-GFP, Thy1, and nuclei overlaid for the entire viewing field and include a magnified view of Thy1 below. Scale bar: 100 μm. FIG. 2B is the experimental scheme, showing that Adult Col1α1-GFP mice (n=5-7/group) were given Bleo (1 U/kg in 100 μL saline) every 12 days for a total of 4 doses by orotracheal intubation/MicroSprayer. The last dose (4^{th}) was administered at day zero. Lung samples were collected at 28 or 56 days after the last Bleo instillation. FIG. 2C is a series of bar graphs of the positively stained area quantified as a total density using ImageJ software (NIH). FIG. 2D is a series of bar graphs of mRNA expression of Thy1 and fibrogenic genes (Col1a1, Col3a1) determined by qPCR of the left lobes of the lung tissue. Results are presented as Mean±SEM. Statistical analysis was performed using one-way ANOVA; *P<0.05 and **P<0.01.

FIGS. 3A-3D show that αvβ3 integrin is persistently activated or upregulated in mice lacking Thy-1 expression. FIG. 3A is a series of immunofluorescence (IF) images for αv integrin and αSMA in lung tissue. FIG. 3B is the experimental scheme, showing that adult wild type (WT) and Thy1^{-/-} mice were given a single bleomycin (Bleo) dose (4 U/kg, in 100 μL saline) by orotracheal intubation/MicroSprayer at day zero. Lung samples were collected at 14, 28, 42, and 56 days after Bleo instillation (n=4-5/group). FIGS. 3C and 3D are bar graphs of quantification of active αvβ3 integrin (WOW-1) and αSMA in lung tissue. Scale bar: 100 Results are presented as Mean±SEM. Two groups were compared using 2-tailed Student's t test, *P<0.05, **P<0.01 versus WT group.

FIGS. 4A-4E show Bleomycin (Bleo) induced acute injury in Thy1^{-/-} and wild type (WT) mice. FIG. 4A is the experimental scheme, showing that mice were induced with a single Bleo dose (4 U/kg in 100 μL saline) or saline by orotracheal intubation/MicroSprayer. Bronchoalveolar lavage fluid (BALF) and lung samples were collected at day 3 (n=6/group). FIG. 4B is a scatter plot of the total protein in BALF. FIG. 4C is a series of hematoxylin and eosin stains. Scale bar: 100 FIGS. 4D and 4E are total cell and differential counts in BALF of Thy1^{-/-} and WT mice. Results are presented as Mean±SEM. Statistical analysis was performed using one-way ANOVA, *P<0.05, **P<0.01 versus controls.

FIGS. 5A-5D show that soluble Thy-1 reverses established Bleomycin (Bleo)-induced lung fibrosis in mice. FIG. 5A is a dosing regimen of a single intratracheal (i.t.) Bleo dose model of lung fibrosis: human sThy1-Fc (1 mg/kg) or PBS was given intravenously (i.v.) to C57BL/6 wild type (WT) mice on day 14 after Bleo (5 U/kg) treatment by orotracheal intubation/MicroSprayer (n=5/group). At 21 days, lung samples were collected and 10% formalin-fixed, paraffin-embedded, and processed. FIG. 5B is hematoxylin and eosin (H&E). Masson's trichrome, and immunohistochemistry αSMA stained slides. Scale bar: 200 FIG. 5C is a plot of fibrosis scores calculated using the H&E slides. FIG. 5D is a plot of a half lung/mouse showing the quantification of hydroxyproline content. Results are presented as Mean±SEM. Statistical analysis was performed using one-way ANOVA; *P<0.05 versus Bleo-PBS group.

FIGS. 6A-6D show that soluble Thy-1 reverses myofibroblastic differentiation of senescent human lung myofibroblasts in a dose-dependent manner in vitro. FIGS. 6A-6D show human lung fibroblasts (CCL-210) incubated with human TGF-01 (10 ng/ml for 48 hours) and subsequently subjected to a 5-day serum-free media to induce senescence (n=4/group, 3 times). FIG. 6A is a series of plots of myofibroblast differentiation and senescence validated by qPCR of Acta2, Col1a1, and p21. FIG. 6B is a series of plots of qPCR for Acta2, Col1a1 after treatment of myofibroblasts with sThy-1-IgG Fc (10, 100, 1,000 ng/mL), Thy-1(RLE)-IgG Fc or IgG Fc, for 48 hrs. FIG. 6C is a Western Blot (WB) of αSMA expression. FIG. 6D is the quantification of the WB of FIG. 6C. Results are presented as Mean±SEM. Statistical analysis was performed using 2-tailed Student's t test and one-way ANOVA; *P<0.05, **P<0.01.

FIGS. 7A-7E show that soluble Thy-1 reverses doxycycline TGF-01-induced lung fibrosis in doxycycline (Dox)-treated CC10-rtTA-tTS-TGF-β1 mice. FIG. 7A is H&E and Trichrome staining, Immunofluorescence (IF) for αv integrin, αSMA, and TGF-β1 in lung tissue. Scale bar: 200 μm. FIG. 7B is the experimental scheme. Mice were randomized to Dox in food (625 mg/kg) and water (1.0 mg/mL) for 4 weeks. Dox induced mice were treated i.v. with a single 1 mg/kg dose of either recombinant human Thy-1-Fc or RLE-mutated Thy-1[Thy-1(RLE)-IgG Fc], IgG-Fc, or saline at day 28. The lungs were assessed one week after treatment (n=4-5/group). FIG. 7C is a plot of hydroxyproline quantification in lung tissue. FIG. 7D is a series of plots of the quantification of αv integrin, αSMA, and TGF-β1 IF staining. FIG. 7E is a series of plots of profibrotic genes (Col1a1, Col3a1, Tgfb1 and Acta2) in lung tissue extracts by qPCR. Results are presented as Mean±SEM. Statistical analysis was performed using one-way ANOVA; *P<0.05, **P<0.01 versus sThy-1 group, ^{##}P<0.01 versus Saline, IgG and sThy-1 (RLE) groups. ^{#}P<0.05 versus Tgfb1 Tg(−) group.

FIG. 8 is a proposed pathway of fibroblastic Thy-1 silencing and sThy-1 addition in lung fibrosis.

### DETAILED DESCRIPTION OF THE INVENTION

The present disclosure is related to the discovery that Thy-1 expression in vivo is reversibly diminished in activated fibroblasts or myofibroblasts in self-resolving fibrosis. However, Thy-1 is silenced in myofibroblasts in progressive fibrosis, which is associated with persistent activation of αv integrin and impaired tissue remodeling characterized by aberrant fibroblast activation and excessive deposition of extracellular matrix. Moreover, targeting of αv integrin by sThy-1, via an integrin-binding RGD-like motif, has therapeutic effectiveness in murine models of bleomycin- and TGF-β1-induced fibrogenesis, indicative of therapeutic potential in reversing established lung fibrosis.

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed. For example, in particular embodiments the amino acid is not A, G or I; is not A; is not G or V; etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

Amino acids are represented herein in the manner recommended by the IUPAC-IUB Biochemical Nomenclature Commission, or (for amino acids) by either the one-letter code, or the three letter code, both in accordance with 37 C.F.R. § 1.822 and established usage.

As used herein, the transitional phrase “consisting essentially of” is to be interpreted as encompassing the recited materials or steps and those that do not materially affect the basic and novel characteristic(s) of the claimed invention (e.g., tissue staining). Thus, the term “consisting essentially of” as used herein should not be interpreted as equivalent to “comprising.”

The term “consists essentially of” (and grammatical variants), as applied to a polypeptide sequence of this invention, means a polypeptide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence such that the function of the polypeptide is not materially altered. The total of ten or less additional amino acids includes the total number of additional amino acids on both ends added together. The term “materially altered,” as applied to polypeptides of the invention, refers to an increase or decrease in enzymatic activity of at least about 50% or more as compared to the activity of a polypeptide consisting of the recited sequence.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof as used with respect to a polypeptide and a fibroblast cell, refers to bringing the polypeptide and the fibroblast cell in sufficiently close proximity to each other for one to exert a biological effect on the other. In some embodiments, the term contact means binding of the polypeptide to the fibroblast cell and/or to ECM to which the fibroblast cell is attached.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical to the reference polypeptide or amino acid sequence. Such a polypeptide fragment according to the invention may be, where appropriate, included in a larger polypeptide of which it is a constituent.

As used herein, the terms “protein” and “polypeptide” are used interchangeably and encompass both peptides and proteins, unless indicated otherwise.

The “N-terminus” of a polypeptide is any portion of the polypeptide that starts from the N-terminal amino acid residue and continues to a maximum of the midpoint of the polypeptide.

The “C-terminus” of a polypeptide is any portion of the polypeptide that starts from the C-terminal amino acid residue and continues to a maximum of the midpoint of the polypeptide.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a peptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-myc, FLAG epitope, an Fc region, etc.

As used herein, a “functional” polypeptide or “functional fragment” is one that substantially retains at least one biological activity normally associated with that polypeptide (e.g., cell adhesion). In particular embodiments, the “functional” polypeptide or “functional fragment” substantially retains all of the activities possessed by the unmodified polypeptide. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as cell adhesion and tissue fibrosis inhibitory activity can be measured using assays that are well known in the art and as described herein.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An isolated cell refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and adenovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992); Wu et al., J. Biol. Chem. 263:14621 (1988); and Hartmut et al., Canadian Patent Application No. 2,012,311, filed Mar. 15, 1990).

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

The term “transfection” or “transduction” means the uptake of exogenous or heterologous nucleic acid (RNA and/or DNA) by a cell. A cell has been “transfected” or “transduced” with an exogenous or heterologous nucleic acid when such nucleic acid has been introduced or delivered inside the cell. A cell has been “transformed” by exogenous or heterologous nucleic acid when the transfected or transduced nucleic acid imparts a phenotypic change in the cell and/or a change in an activity or function of the cell. The transforming nucleic acid can be integrated (covalently linked) into chromosomal DNA making up the genome of the cell or it can be present as a stable plasmid.

### Soluble Thy-1 Polypeptide

One aspect of the invention relates to products that can be used to carry out the methods disclosed herein. Thus, one aspect of the invention relates to a soluble Thy-1 polypeptide or a functional fragment thereof. In some embodiments, the polypeptide is human Thy-1 polypeptide or a functional fragment thereof. In some embodiments, the soluble Thy-1 polypeptide or a functional fragment thereof comprises at least the integrin binding domain of Thy-1, e.g., including the RLD (RGD-like motif). In some embodiments, the soluble Thy-1 polypeptide or a functional fragment thereof comprises at least the syndecan-4 binding domain of Thy-1. In some embodiments, the soluble Thy-1 polypeptide or a functional fragment thereof comprises at least integrin binding domain and the syndecan-4 binding domain of Thy-1.

In some embodiments, the Thy-1 polypeptide or a functional fragment thereof comprises, consists essentially of, or consists of the publicly known amino acid sequence of the Thy-1 protein (e.g., as disclosed in GenBank and disclosed herein). For example, the human Thy-1 sequence is disclosed in GenBank Accession Numbers AAA61180.1, ACE08960.1, ACH06703.1, ACH16792.1, QTV75171.1. In another embodiment, the Thy-1 polypeptide or a functional fragment thereof comprises, consists essentially of, or consists of an amino acid sequence that is at least 70% identical, e.g., at least 75%, 80%, 85%, 90%, 95%, 96%, 97%, 98%, or 99% identical to the publicly known amino acid sequence.

The amino acid sequence of human Thy-1 (SEQ ID NO:1) is disclosed below. The conserved RLD (RGD-like motif), which may be important for activity is underlined.

```
Thy-1
(SEQ ID NO: 1)
    1 MNLAISIALL LTVLQVSRGQ KVTSLTACLV DQSLRLDCRH ENTSSSPIQY EFSLTRETKK
  61 HVLFGTVGVP EHTYRSRTNF TSKYHMKVLY LSAFTSKDEG TYTCALHHSG HSPPISSQNV
121 TVLRDKLVKC EGISLLAQNT SWLLLLLLSL SLLQATDFMS L
```

The polypeptide of the invention also includes functional portions or fragments of Thy-1. The length of the fragment is not critical as long as it substantially retains the biological activity of the polypeptide (e.g., integrin binding or antifibrotic activity). Illustrative fragments comprise at least about 4, 6, 8, 10, 12, 15, 20, 25, 30, 35, 40, 45, 50, 75, 100, 150, 200, or more contiguous amino acids of a Thy-1 protein. In other embodiments, the fragment comprises no more than about 200, 150, 100, 75, 50, 45, 40, 35, 30, 25, 20, 15, 12, 10, 8, 6, or 4 contiguous amino acids of a Thy-1 polypeptide.

In some embodiments, the GPI attachment signal on Thy-1 is deleted, which may help form a soluble form of Thy-1. In some embodiments, the GPI attachment signal comprises the last 31 amino acids of SEQ ID NO: 1.

Likewise, those skilled in the art will appreciate that the present invention also encompasses fusion polypeptides comprising a Thy-1 polypeptide or a functional fragment thereof. The Thy-1 polypeptide may be fused at its N-terminus or C-terminus to the other polypeptide. For example, it may be useful to express the polypeptide (or functional fragment) as a fusion protein that can be recognized by a commercially available antibody (e.g., FLAG motifs) or as a fusion protein that can otherwise be more easily purified (e.g., by addition of a poly-His tail). Additionally, fusion proteins that enhance the stability of the polypeptide may be produced, e.g., fusion proteins comprising maltose binding protein (MBP) or glutathione-S-transferase. In some embodiments, the Thy-1 polypeptide is fused to an immunoglobulin Fc region. In other embodiments, the fusion protein does not comprise an immunoglobulin Fc region. As another alternative, the fusion protein can comprise a reporter molecule. In other embodiments, the fusion protein can comprise a polypeptide that provides a function or activity that is the same as or different from the activity of the polypeptide, e.g., a targeting, binding, or enzymatic activity or function.

Likewise, it will be understood that the polypeptides specifically disclosed herein will typically tolerate substitutions in the amino acid sequence and substantially retain biological activity. To identify polypeptides of the invention other than those specifically disclosed herein, amino acid substitutions may be based on any characteristic known in the art, including the relative similarity or differences of the amino acid side-chain substituents, for example, their hydrophobicity, hydrophilicity, charge, size, and the like.

One aspect of the invention relates to a polynucleotide encoding the polypeptide of the invention. In embodiments of the invention, the polynucleotide encoding the polypeptide will hybridize to the nucleic acid sequences encoding Thy-1 proteins that are known in the art or fragments thereof under standard conditions as known by those skilled in the art and encode a functional polypeptide or functional fragment thereof.

For example, hybridization of such sequences may be carried out under conditions of reduced stringency, medium stringency or even stringent conditions (e.g., conditions represented by a wash stringency of 35-40% formamide with 5×Denhardt's solution, 0.5% SDS and 1×SSPE at 37° C.; conditions represented by a wash stringency of 40-45% formamide with 5×Denhardt's solution, 0.5% SDS, and 1×SSPE at 42° C.; and conditions represented by a wash stringency of 50% formamide with 5×Denhardt's solution, 0.5% SDS and 1×SSPE at 42° C., respectively) to the polynucleotide sequences encoding the Thy-1 protein or functional fragments thereof specifically disclosed herein. See, e.g., Sambrook et al., Molecular Cloning: A Laboratory Manual 4^{th} Ed. (Cold Spring Harbor, N Y, 2012).

In other embodiments, polynucleotide sequences encoding the polypeptide have at least about 70%, 80%, 85%, 90%, 95%, 96%, 97%, 98%, 99% or higher sequence identity with the publicly known nucleic acid sequences (disclosed in GenBank) or functional fragments thereof and encode a functional polypeptide or functional fragment thereof.

Further, it will be appreciated by those skilled in the art that there can be variability in the polynucleotides that encode the polypeptides (and fragments thereof) of the present invention due to the degeneracy of the genetic code. The degeneracy of the genetic code, which allows different nucleic acid sequences to code for the same polypeptide, is well known in the literature.

As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity with respect to the coding sequence of the polypeptides disclosed herein is defined as the percentage of nucleotide residues in the candidate sequence that are identical with the nucleotides in the polynucleotide.

The alignment may include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Those skilled in the art will appreciate that the isolated polynucleotides encoding the polypeptides of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

To illustrate, the polypeptide coding sequence can be operatively associated with a cytomegalovirus (CMV) major immediate-early promoter, an albumin promoter, an Elongation Factor 1-α (EF1-α) promoter, a PγK promoter, a MFG promoter, or a Rous sarcoma virus promoter.

Inducible promoter/enhancer elements include hormone-inducible and metal-inducible elements, and other promoters regulated by exogenously supplied compounds, including without limitation, the zinc-inducible metallothionein (MT) promoter; the dexamethasone (Dex)-inducible mouse mammary tumor virus (MMTV) promoter; the T7 polymerase promoter system (see WO 98/10088); the ecdysone insect promoter (No et al., Proc. Natl. Acad. Sci. USA 93:3346 (1996)); the tetracycline-repressible system (Gossen et al., Proc. Natl. Acad. Sci. USA 89:5547 (1992)); the tetracycline-inducible system (Gossen et al., Science 268:1766 (1995); see also Harvey et al., Curr. Opin. Chem. Biol. 2:512 (1998)); the RU486-inducible system (Wang et al., Nat. Biotech. 15:239 (1997); Wang et al., Gene Ther., 4:432 (1997)); and the rapamycin-inducible system (Magari et al., J. Clin. Invest. 100:2865 (1997)).

Moreover, specific initiation signals are generally required for efficient translation of inserted polypeptide coding sequences. These translational control sequences, which can include the ATG initiation codon and adjacent sequences, can be of a variety of origins, both natural and synthetic.

The present invention further provides cells comprising the isolated polypeptides of the invention. The cell may be a cultured cell or a cell in vivo, e.g., for use in therapeutic methods, diagnostic methods, screening methods, methods for studying the biological action of the Thy-1 protein, etc. In another embodiment, the cell is an ex vivo cell that has been isolated from a subject. The ex vivo cell may be modified and then reintroduced into the subject for diagnostic or therapeutic purposes.

For expression of the polypeptides of the invention, the isolated polynucleotide can be incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a polypeptide operatively associated with the promoter, and, optionally, a termination sequence including a stop signal for RNA polymerase and a polyadenylation signal for polyadenylase.

Non-limiting examples of promoters of this invention include CYC1, HIS3, GAL1, GAL4, GAL10, ADH1, PGK, PHO5, GAPDH, ADC1, TRP1, URA3, LEU2, ENO, TPI, and alkaline phosphatase promoters (useful for expression in Saccharomyces); AOX1 promoter (useful for expression in Pichia); β-lactamase, lac, ara, tet, trp, IP_{L}, IP_{R}, T7, tac, and trc promoters (useful for expression in Escherichia coli); light regulated-, seed specific-, pollen specific-, ovary specific-, pathogenesis or disease related-promoters, cauliflower mosaic virus 35S, CMV 35S minimal, cassaya vein mosaic virus (CsVMV), chlorophyll a/b binding protein, ribulose 1,5-bisphosphate carboxylase, shoot-specific promoters, root specific promoters, chitinase, stress inducible promoters, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells).

Further examples of animal and mammalian promoters known in the art include, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, baculovirus IE1 promoter, elongation factor 1 alpha (EF1) promoter, phosphoglycerate kinase (PGK) promoter, ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis and/or disease-related promoters, and promoters that exhibit tissue specificity, such as the elastase I gene control region, which is active in pancreatic acinar cells; the insulin gene control region active in pancreatic beta cells, the immunoglobulin gene control region active in lymphoid cells, the mouse mammary tumor virus control region active in testicular, breast, lymphoid and mast cells; the albumin gene promoter, the Apo AI and Apo AII control regions active in liver, the alpha-fetoprotein gene control region active in liver, the alpha 1-antitrypsin gene control region active in the liver, the beta-globin gene control region active in myeloid cells, the myelin basic protein gene control region active in oligodendrocyte cells in the brain, the myosin light chain-2 gene control region active in skeletal muscle, and the gonadotropic releasing hormone gene control region active in the hypothalamus, the pyruvate kinase promoter, the villin promoter, the promoter of the fatty acid binding intestinal protein, the promoter of smooth muscle cell α-actin, and the like. In addition, any of these expression sequences of this invention can be modified by addition of enhancer and/or regulatory sequences and the like.

Enhancers that may be used in embodiments of the invention include but are not limited to: an SV40 enhancer, a cytomegalovirus (CMV) enhancer, an elongation factor I (EF1) enhancer, yeast enhancers, viral gene enhancers, and the like.

Termination control regions, i.e., terminator or polyadenylation sequences, may be derived from various genes native to the preferred hosts. In some embodiments of the invention, the termination control region may comprise or be derived from a synthetic sequence, a synthetic polyadenylation signal, an SV40 late polyadenylation signal, an SV40 polyadenylation signal, a bovine growth hormone (BGH) polyadenylation signal, viral terminator sequences, or the like.

It will be apparent to those skilled in the art that any suitable vector can be used to deliver the polynucleotide to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Suitable vectors include plasmid vectors, viral vectors (e.g., retrovirus, alphavirus; vaccinia virus; adenovirus, adeno-associated virus and other parvoviruses, lentivirus, poxvirus, or herpes simplex virus), lipid vectors, poly-lysine vectors, synthetic polyamino polymer vectors, and the like.

Any viral vector that is known in the art can be used in the present invention. Protocols for producing recombinant viral vectors and for using viral vectors for nucleic acid delivery can be found in Ausubel et al., Current Protocols in Molecular Biology (Green Publishing Associates, Inc. and John Wiley & Sons, Inc., New York) and other standard laboratory manuals (e.g., Vectors for Gene Therapy. In: Current Protocols in Human Genetics. John Wiley and Sons, Inc.: 1997).

Non-viral transfer methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. For example, naked plasmids can be introduced into fibroblast cells by injection into the tissue. Expression can extend over many months, although the number of positive cells is typically low (Wolff et al., Science 247:247 (1989)). Cationic lipids have been demonstrated to aid in introduction of nucleic acids into some cells in culture (Felgner and Ringold, Nature 337:387 (1989)). Injection of cationic lipid plasmid DNA complexes into the circulation of mice has been shown to result in expression of the DNA in lung (Brigham et al., Am. J. Med. Sci. 298:278 (1989)). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., No Shinkei Geka 20:547 (1992); PCT publication WO 91/06309; Japanese patent application 1047381; and European patent publication EP-A-43075).

Liposomes that consist of amphiphilic cationic molecules are useful as non-viral vectors for nucleic acid delivery in vitro and in vivo (reviewed in Crystal, Science 270:404 (1995); Blaese et al., Cancer Gene Ther. 2:291 (1995); Behr et al., Bioconjugate Chem. 5:382 (1994); Remy et al., Bioconjugate Chem. 5:647 (1994); and Gao et al., Gene Therapy 2:710 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84:7413 (1987); Loeffler et al., Meth. Enzymol. 217:599 (1993); Felgner et al., J. Biol. Chem. 269:2550 (1994)).

Several groups have reported the use of amphiphilic cationic lipid:nucleic acid complexes for in vivo transfection both in animals and in humans (reviewed in Gao et al., Gene Therapy 2:710 (1995); Zhu et al., Science 261:209 (1993); and Thierry et al., Proc. Natl. Acad. Sci. USA 92:9742 (1995)). U.S. Pat. No. 6,410,049 describes a method of preparing cationic lipid:nucleic acid complexes that have a prolonged shelf life.

Expression vectors can be designed for expression of polypeptides in prokaryotic or eukaryotic cells. For example, polypeptides can be expressed in bacterial cells such as E. coli, insect cells (e.g., the baculovirus expression system), yeast cells, plant cells or mammalian cells. Some suitable host cells are discussed further in Goeddel, Gene Expression Technology: Methods in Enzymology 185, Academic Press, San Diego, Calif. (1990). Examples of bacterial vectors include pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, and pRIT5 (Pharmacia). Examples of vectors for expression in the yeast S. cerevisiae include pYepSecl (Baldari et al., EMBO J. 6:229 (1987)), pMFa (Kurjan and Herskowitz, Cell 30:933 (1982)), pJRY88 (Schultz et al., Gene 54:113 (1987)), and pYES2 (Invitrogen Corporation, San Diego, Calif.). Baculovirus vectors available for expression of nucleic acids to produce proteins in cultured insect cells (e.g., Sf 9 cells) include the pAc series (Smith et al., Mol. Cell. Biol. 3:2156 (1983)) and the pVL series (Lucklow and Summers Virology 170:31 (1989)).

Examples of mammalian expression vectors include pWLNEO, pSV2CAT, pOG44, pXT1, pSG (Stratagene) pSVK3, PBPV, pMSG, PSVL (Pharmacia), pCDM8 (Seed, Nature 329:840 (1987)) and pMT2PC (Kaufman et al., EMBO J. 6:187 (1987)). When used in mammalian cells, the expression vector's control functions are often provided by viral regulatory elements. For example, commonly used promoters are derived from polyoma, adenovirus 2, cytomegalovirus and Simian Virus 40.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

In addition to the regulatory control sequences discussed above, the recombinant expression vector can contain additional nucleotide sequences. For example, the recombinant expression vector can encode a selectable marker gene to identify host cells that have incorporated the vector.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA and RNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al., Molecular Cloning: A Laboratory Manual 4^{th} Ed. (Cold Spring Harbor, N Y, 2012), and other laboratory manuals.

If stable integration is desired, often only a small fraction of cells (in particular, mammalian cells) integrate the foreign DNA into their genome. In order to identify and select integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

### Methods of Using Soluble Thy-1

One aspect of the invention relates to a method of reversing profibrotic activation of a fibroblast comprising contacting the fibroblast with an effective amount of a soluble Thy-1 polypeptide or a functional fragment thereof or a fusion protein comprising a soluble Thy-1 polypeptide or a functional fragment thereof.

Another aspect of the invention relates to a method of treating, inhibiting, and/or reversing tissue fibrosis in a subject in need thereof, comprising delivering to the subject a therapeutically effective amount of a soluble Thy-1 polypeptide or a functional fragment thereof or a fusion protein comprising a soluble Thy-1 polypeptide or a functional fragment thereof, thereby treating, inhibiting, and/or reversing tissue fibrosis in the subject.

In some embodiments, the administration of a therapeutically effective amount of a soluble Thy-1 polypeptide or a functional fragment thereof or a fusion protein comprising a soluble Thy-1 polypeptide or a functional fragment thereof blocks the initiation of tissue fibrosis. In other embodiments, the administration of a therapeutically effective amount of a soluble Thy-1 polypeptide or a functional fragment thereof or a fusion protein comprising a soluble Thy-1 polypeptide or a functional fragment thereof inhibits the progression of tissue fibrosis. In other embodiments, the administration of a therapeutically effective amount of a soluble Thy-1 polypeptide or a functional fragment thereof or a fusion protein comprising a soluble Thy-1 polypeptide or a functional fragment thereof reverses existing tissue fibrosis. In other embodiments, the administration of a therapeutically effective amount of a soluble Thy-1 polypeptide or a functional fragment thereof or a fusion protein comprising a soluble Thy-1 polypeptide or a functional fragment thereof accelerates the resolution of tissue fibrosis.

The tissue fibrosis can involve any tissue or organ. In some embodiments, the tissue fibrosis is lung fibrosis, optionally idiopathic lung fibrosis. In other embodiments, the fibrosis is kidney, liver, or heart fibrosis. In some embodiments, the fibrosis is due to arthritis and is present, e.g., in a joint or other tissue. In some embodiments, the tissue fibrosis is self-resolving. In other embodiments, the tissue fibrosis is non-resolving.

In some embodiments, the tissue fibrosis is due to an acute injury. For example, tissue fibrosis may develop from chemical exposure, surgery, inflammation, and/or burns. In other embodiments, the tissue fibrosis is due to a chronic condition and/or exposure. For example, chronic chemical or irritant exposure including radiation and chemotherapy may lead to fibrosis.

One aspect of the invention relates to a method of inhibiting alveolar septal thickening, comprising contacting alveolar cells with the polypeptide or a functional fragment thereof of the invention, thereby inhibiting alveolar septal thickening. Inhibition of alveolar septal thickening can be measured by any technique known in the art or disclosed herein. Inhibition of alveolar septal thickening is measured relative to the level of alveolar septal thickening in the absence of contact with the polypeptide or a functional fragment thereof of the invention. In some embodiments, alveolar septal thickening is inhibited by at least about 10%, e.g., at least about 10%, 20%, 30%, 40%, 50%, 60%, 70%, 80%, 90%, or more.

An additional aspect of the invention relates to a treating or preventing a disorder responsive to inhibition of tissue fibrosis in a subject in need thereof, comprising delivering to the subject a therapeutically effective amount of the polypeptide or a functional fragment thereof or pharmaceutical composition of the invention, thereby treating or preventing the disorder. As used herein, the term “disorder responsive to inhibition of tissue fibrosis,” refers to any disease, disorder, or condition that can be treated and/or prevented, or at least one symptom thereof reduced, by inhibiting or reversing tissue fibrosis. The disorder or condition in the methods of the invention can be, in non-limiting examples, fibrosis of an organ or tissue such as lung, kidney, heart, liver, or joint, fibrosis formation associated with surgery, fibrosis formation around biological implants, and fibrosis formation associated with radiation therapy.

The polypeptide or a functional fragment thereof of the present invention can optionally be delivered in conjunction with other therapeutic agents. The additional therapeutic agents can be delivered concurrently with the polypeptide or a functional fragment thereof of the invention. As used herein, the word “concurrently” means sufficiently close in time to produce a combined effect (that is, concurrently can be simultaneously, or it can be two or more events occurring within a short time period before or after each other). In one embodiment of the invention, the polypeptide or a functional fragment thereof is delivered to a patient concurrently with a compound that treats and/or prevents tissue fibrosis, e.g., pirfenidone or nintedanib. In some embodiments, the combined activity of the polypeptide or a functional fragment thereof and the other therapeutic agent is superior to the other therapeutic agent alone.

### Subjects, Pharmaceutical Formulations, and Modes of Administration

Polypeptides or functional fragments thereof according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles and adults.

In particular embodiments, the present invention provides a pharmaceutical composition comprising a polypeptide or functional fragment thereof of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

The formulations of the invention can optionally comprise medicinal agents, pharmaceutical agents, carriers, adjuvants, dispersing agents, diluents, and the like.

The polypeptides or functional fragments thereof of the invention can be formulated for administration in a pharmaceutical carrier in accordance with known techniques. See, e.g., Remington, The Science And Practice of Pharmacy (23^{rd} Ed. 2020). In the manufacture of a pharmaceutical formulation according to the invention, the polypeptide or a functional fragment thereof (including the physiologically acceptable salts thereof) is typically admixed with, inter alia, an acceptable carrier. The carrier can be a solid or a liquid, or both, and may be formulated with the polypeptide or a functional fragment thereof as a unit-dose formulation, for example, a metered dose inhaler, which can contain from 0.01 or 0.5% to 95% or 99% by weight of the polypeptide or a functional fragment thereof. One or more polypeptide or a functional fragment thereof can be incorporated in the formulations of the invention, which can be prepared by any of the well-known techniques of pharmacy.

Another aspect of the invention relates to a kit comprising the polypeptide or a functional fragment thereof of the invention and useful for carrying out the methods of the invention. The kit may further comprise additional reagents for carrying out the methods (e.g., buffers, containers, additional therapeutic agents) as well as instructions.

One aspect of the present invention is a method of contacting a polypeptide or functional fragment thereof to a cell in vitro. The polypeptide or functional fragment thereof may be contacted with the cells at the appropriate concentration according to standard methods suitable for the particular target cells. Concentrations of the polypeptide or functional fragment thereof to administer can vary, depending upon the target cell type and number, and can be determined by those of skill in the art without undue experimentation. In representative embodiments, at least about 10, 100, and 1,000 ng/mL, are contacted with the cell.

The cell(s) with which the polypeptide or functional fragment thereof is contacted can be of any type. Moreover, the cell can be from any species of origin, as indicated above.

The polypeptide or functional fragment thereof can be contacted with cells in vitro for the purpose of administering the modified cell to a subject, e.g., for secretion of the polypeptide or functional fragment thereof from the modified cell. In particular embodiments, the cells have been removed from a subject, the polypeptide or functional fragment thereof is contacted therewith, and the cells are then administered back into the subject. Methods of removing cells from subject for manipulation ex vivo, followed by introduction back into the subject are known in the art (see, e.g., U.S. Pat. No. 5,399,346). In particular embodiments, the cells with the polypeptide or functional fragment thereof are administered to the subject in a treatment effective or prevention effective amount in combination with a pharmaceutical carrier.

In some embodiments, Thy-1 is delivered to a cell in vitro or ex vivo by contacting the cell with an effective amount of the polypeptide or functional fragment thereof, thereby delivering Thy-1 to the cell. In some embodiments, the cells are then transplanted to a subject in need thereof.

A further aspect of the invention is a method of administering the polypeptide or functional fragment thereof to subjects. Administration of the polypeptide or functional fragment thereof according to the present invention to a human subject or an animal in need thereof can be by any means known in the art. Optionally, the polypeptide or functional fragment thereof is delivered in a treatment effective or prevention effective dose in a pharmaceutically acceptable carrier.

Dosages of the polypeptide or functional fragment thereof to be administered to a subject depend upon the mode of administration, the disease or condition to be treated and/or prevented, the individual subject's condition, and the like, and can be determined in a routine manner. Exemplary doses for achieving therapeutic effects are doses that achieve in vivo concentrations of at least about 10, 100, and 1,000 ng/mL.

In particular embodiments, more than one administration (e.g., two, three, four or more administrations) may be employed to achieve the desired level of expression over a period of various intervals, e.g., daily, weekly, monthly, yearly, etc.

In particular embodiments, a polypeptide or functional fragment thereof according to the present invention is administered to the subject to treat, inhibit, and/or reverse tissue fibrosis.

In another aspect, the invention further encompasses a method of treating, inhibiting, and/or reversing tissue fibrosis in a subject in need thereof, comprising administering to the subject a therapeutically effective amount of a polypeptide or functional fragment thereof that has Thy-1 activity, thereby treating, inhibiting or reversing tissue fibrosis in the subject.

In some embodiments, the polypeptide or functional fragment thereof is administered to the subject by injection. In other embodiments, the polypeptide or functional fragment thereof is administered to the subject orally or topically. In the methods of the invention, the subject may be one has been diagnosed with tissue fibrosis or is suspected of having tissue fibrosis. Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intro-lymphatic, and the like, as well as direct tissue or organ injection (e.g., to liver, skeletal muscle, cardiac muscle, diaphragm muscle or kidney). The most suitable route in any given case will depend on the nature and severity of the condition being treated.

In one embodiment, the polypeptides or fragments thereof of the invention are administered directly to a subject. Generally, the compounds of the invention will be suspended in a pharmaceutically-acceptable carrier (e.g., physiological saline) and administered orally or by intravenous infusion, or administered subcutaneously, intramuscularly, intrathecally, intraperitoneally, intrarectally, intravaginally, intranasally, intragastrically, intratracheally, or intrapulmonarily. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as lungs, kidney, or intestines. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of polypeptides and fragments thereof available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the polypeptides and fragments thereof in a suitable delivery vehicle (e.g., polymeric microparticles or implantable devices) may increase the efficiency of delivery, particularly for oral delivery.

According to certain embodiments, the polypeptide or a functional fragment thereof can be targeted to specific cells or tissues in vivo. Targeting delivery vehicles, including liposomes and targeted systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262:13973 (1987); and U.S. Pat. No. 4,957,735 to Huang et al., each of which is incorporated herein by reference in its entirety).

An additional aspect of the invention relates to a dosage delivery device comprising the pharmaceutical composition. In some embodiments, the dosage delivery device is an inhaler for delivery of the composition to the airways of a subject, e.g., by oral and/or nasal inhalation.

Injectables can be prepared in conventional forms, either as liquid solutions or suspensions, solid forms suitable for solution or suspension in liquid prior to injection, or as emulsions. Alternatively, one may administer the polypeptide or functional fragment thereof of the invention in a local manner, for example, in a depot or sustained-release formulation. Further, the polypeptide or functional fragment thereof can be delivered adhered to a surgically implantable matrix.

Having described the present invention, the same will be explained in greater detail in the following examples, which are included herein for illustration purposes only, and which are not intended to be limiting to the invention.

### Example 1

### Materials and Methods

Cell Culture: Human lung fibroblasts (CCL-210) (50,000 cells) were cultured in 6-well plates in DMEM with high glucose, 10% fetal bovine serum, and 1% penicillin-streptomycin, and confluent cells were passaged by 0.25% trypsin. To rule out serum effects, fibroblasts were incubated in serum-deprived media for 16 hours prior to TGF-β1 treatment. By treating with TGF-β1 in serum-free media (10 ng/mL) for 48 hours, differentiation into myofibroblasts was induced. TGFβ1-induced myofibroblasts were subjected to FBS 0% or 20% media to model myofibroblast senescence. Then cells were treated with indicated concentration of soluble Thy-1 (10, 100, 1000 ng/mL) for 48 hours to test dedifferentiation from myofibroblasts. Human IgG-FC (Enzo Life Science, ALX-203-005-2060), sThy-1 (RLE)-FC (Enzo Life Science, ALX-522-097-0050), and sThy-1-FC (Enzo Life Science, ALX-522-091-0050) were reconstituted in PBS before use. IgG-Fc and sThy-1 RLE-Fc concentrations were 1000 ng/mL. PBS treatment without TGF-β1 and sThy1 treatment was used as a negative control.

Experimental Animals: Mice with constitutive knockout of Thy-1 (Thy1^{-/-}), as well as Col-GFP (expressing collagen-a1(I) promoter/enhancer-driven GFP) mice have been previously described. Thy1^{-/-} Col-GFP mice were generated by crossing of Thy1^{-/-} and Col1α1-GFP mice. CC10-rtTA-tTS-Tgfb1 mice, a triple transgenic system, allows bioactive hTGF-β1 to be expressed conditionally in the lung via Doxycycline (Dox) induction. Genotyping of all mice was performed by PCR.

To generate pulmonary fibrosis, WT, Thy1^{-/-}, Col1a1-GFP, and Thy1^{-/-} Col-GFP mice (both genders, 24-week-old) were anesthetized with intraperitoneal injections of ketamine and xylazine (100 and 10 mg/kg body mass, respectively). Mice were given a single (4 U/kg) or repetitive (1 U/kg, each 10 days for 4 times) intratracheal instillation of bleomycin sulphate (4 U/kg body weight, dissolved in 100 μL sterile saline, McKesson) or sterile saline on day 0 using MicroSprayer MS-IA-1C (Penn-Century). Following the instillation, mice were allowed to be monitored daily for mortality. To conditionally induce pulmonary fibrosis, CC10-rtTA-tTS-Tgfb1 mice, both genders, 24-weeks-old, were randomized to food containing Dox (625 mg/kg) and water with Dox (1.0 mg/mL), or normal food and water for 4 weeks.

sThy-1 Treatment: Dox- and bleomycin-induced mice were i.v., treated with a 1000 μg/kg of either recombinant human Thy-1-Fc or RLE-mutated Thy-1[Thy-1(RLE)-IgG Fc], IgG-FC and saline as controls separately. The lungs were assessed one week after treatment by histopathology (stained H&E and Trichrome), collagen content (measuring hydroxyproline), profibrotic genes Col1a1, Col3a1, mTgfb1 in the lung tissue extracts by qPCR. αSMA and αv integrin in lung slides were determined by immunofluorescence.

Histopathological Analysis: Lungs were fixed (10% formalin at a constant pressure of 20 cm H₂O), and paraffin-embedded. Five μm-thick sagittal sections of all lobes of fixed lungs were cut and subsequently stained with H&E and Masson's trichrome to evaluate histopathologic changes. Severity of fibrosis was quantified from H&E stained entire lungs using the Ashcroft scoring system. The degree of fibrosis was graded from 0 (normal lung) to 8 (severe distortion of structure, large fibrous areas, and honeycomb lesions). The mean score from all fields (magnification ×200, average 30 fields/animal) was taken as the fibrosis score.

Immunofluorescence: Lungs were sectioned at 6 μm (paraffin embedding) or 10 μm (OCT embedding) followed by double immunofluorescence (IF) with antibodies. Briefly, lung slides were fixed with 3.7% formaldehyde for 10 minutes, permeabilized with 0.25% Triton X-100 (Fisher Scientific) for 8 minutes, blocked with 1% BSA for 1 hour. Lung sections were incubated with primary antibodies, Thy-1 (1:300), WOW-1 (1:200) αSMA (1:300), αv integrin (CD51) (1:200), TGF-β1 (1:200) at 4° C. overnight. After the sections were washed with PBS, Alexa-Fluor-coupled secondary antibodies (1:1000), Goat anti-rabbit IgG (H+L), Texas Red (1:250) were used as secondary antibodies. Nuclear staining was carried out with DAPI using ProLong Diamond Antifade Mountant medium. Fluorescence images were captured on a BZ-X700 microscope. To determine the fluorescence signal in tissue sections, fluorescent cells in five randomly different high-power fields from each slide were quantified.

Hydroxyproline Quantification: Hydroxyproline content was measured using a hydroxyproline assay kit from Biovision according to the manufacture's instruction with slight modification. In brief, whole lungs were homogenized in dH₂O, using 100 μL dH₂O for every 10 mg of tissue. To 100 μL of tissue homogenate, 200 μL concentrated HCl (6N) was added in a pressure-tight, teflon capped vial, and the mixture was hydrolyzed at 120° C. for 3 hours, followed by filtration through a 45 μm syringe filter (Millipore). 10 μL of hydrolyzed sample was transferred to a 96-well plate and was evaporated to dryness under vacuum, to which 100 μL Chloramine T reagent was added per well. After incubation at room temperature for 5 min, 100 μL p-dimethylaminobenzaldehyde reagent was added to each well and further incubated for 90 min at 60° C. Absorbance was measured at 560 nm in a microplate reader.

Western Blot: Cultured cells were homogenized in RIPA tissue lysis buffer plus protease and phosphatase inhibitor cocktail. Equal protein amounts of each lysate were separated on SDS-polyacrylamide gels by electrophoresis before being transferred to polyvinylidene difluoride membranes by electroblotting. Membranes were blocked in TBST with 5% nonfat dry milk and incubated with antibodies αSMA (1:1000), GAPDH (1:1000) at 4° C. with constant rocking overnight. Bound primary antibodies were visualized using appropriate secondary antibody with conjugated horseradish peroxidase and enhanced chemiluminescence reagent.

Quantitative RT-PCR Analysis: A lung lobe was homogenized in 1 mL Trizol reagent. Total RNA was isolated and cDNA synthesized commercially. Real-time RT-PCR was performed using iTaq™ Universal SYBR Green Supermix and using CFX96 real-time PCR detection system. Primers were designed using Beacon Designer and are listed in Table 1. Assays for each sample and primer set were performed in duplicate, with each reaction using 20 ng of cDNA and 10 μM primers in a total reaction volume of 20 μL. Thermal cycling conditions were 95° C. for 30 sec, 35 cycles of 95° C. for 45 sec, 57° C. for 30 sec and 72° C. for 60 sec. The relative quantification of gene expression was determined using the comparative CT method. Actin or B2m were used to normalize the expression data. Comparative threshold (ΔΔCt) was calculated to determine the changes of gene expression. Individual lung cDNA samples (n=3-6) were analyzed for the relative expression of mouse Thy1, Col1a1, Col3a1, Acta2, and Tgfb1.

Statistical Analysis: Statistical analysis was performed using GraphPad Prism 7M. Values are expressed as mean±SEM. The statistical differences were calculated using 2-tailed Student's t test between two groups and 1-way ANOVA for multiple comparisons. P<0.05 was considered statistically significant.

TABLE 1

| Gene | | |
|---|---|---|
| Name | Forward | Reverse |
| Thy1 | 5′-GGGCGACTACTTTTGTGAGC-3′ | 5′-TCTGAACCAGCAGGCTTATG-3′ |
| | (SEQ ID NO: 2) | (SEQ ID NO: 3) |
| Colla1 | 5′-ACATGTTCAGCTTTGTGGACC-3′ | 5′-TAGGCCATTGTGTATGCAGC-3′ |
| | (SEQ ID NO: 4) | (SEQ ID NO: 5) |
| Col3a1 | 5′-ATTGCTGGGATCACTGGAGCAC-3′ | 5′-CCTGGTTTCCCACTTTCACCCTTG-3′ |
| | (SEQ ID NO: 6) | (SEQ ID NO: 7) |
| Acta2 | 5′-ACTGGGACGACATGGAAAAG-3′ | 5′-GTTCAGTGGTGCCTCTGTCA-3′ |
| | (SEQ ID NO: 8) | (SEQ ID NO: 9) |
| Tgfb1 | 5′-GGAGAGCCCTGGATACCAAC-3′ | 5′-CAACCCAGGTCCTTCCTAAA-3′ |
| | (SEQ ID NO: 10) | (SEQ ID NO: 11) |
| Actb | 5′-CTAAGGCCAACCGTGAAAAGAT-3′ | 5′-CACAGCCTGGATGGCTACGT-3′ |
| | (SEQ ID NO: 12) | (SEQ ID NO: 13) |
| B2m | 5′-ATCGAGACATGTGATCAAGC-3′ | 5′-GCGTGCATAAATTGTATAGC-3′ |
| | (SEQ ID NO: 14) | (SEQ ID NO: 15) |

### Example 2

Loss of Thy-1 expression in fibroblasts correlates with lung fibrogenesis, however, the clinical relevance of therapeutic targeting of myofibroblasts via Thy-1-associated pathways remains to be explored. Using single (self-resolving) vs. repetitive (non-resolving) intratracheal administration of bleomycin in type 1 collagen-GFP reporter mice, it was discovered that Thy-1 surface expression, but not mRNA, is reversibly diminished in activated fibroblasts and myofibroblasts in self-resolving fibrosis. However, Thy-1 mRNA expression is silenced in lung with non-resolving fibrosis following repetitive bleomycin, associated with persistent activation of αv integrin. Thy1 null mice showed progressive αv integrin activation and myofibroblast accumulation after a single dose of bleomycin. In vitro, targeting of αv integrin by soluble Thy-1-Fc (sThy-1), but not RLE-mutated Thy-1 or IgG, reversed TGF-β1 induced myofibroblast differentiation in a dose-dependent manner, suggesting that Thy-1's integrin-binding RGD-motif is required for the reversibility of myofibroblast differentiation. In vivo, treatment of established fibrosis induced either by single dose bleomycin in wild type mice or by induction of active TGF-β1 by doxycycline in CC10-rtTA-tTS-TGF-β1 mice with sThy-1 (1000 ng/kg, i.v.) promoted resolution of fibrosis. Collectively, these findings demonstrate that sThy-1 therapeutically inhibits the αv integrin-driven feedback loop that amplifies and sustains fibrosis.

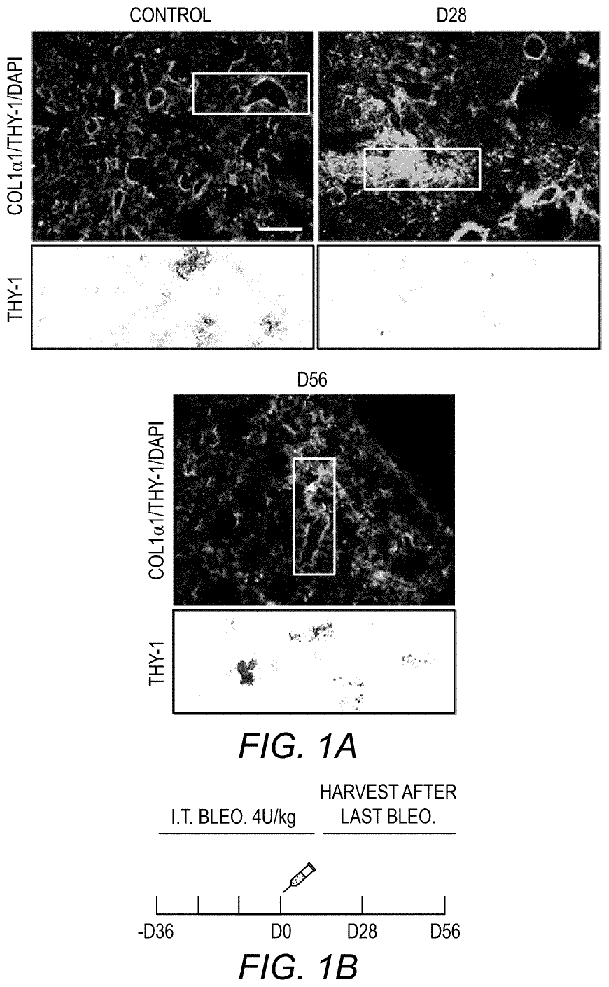

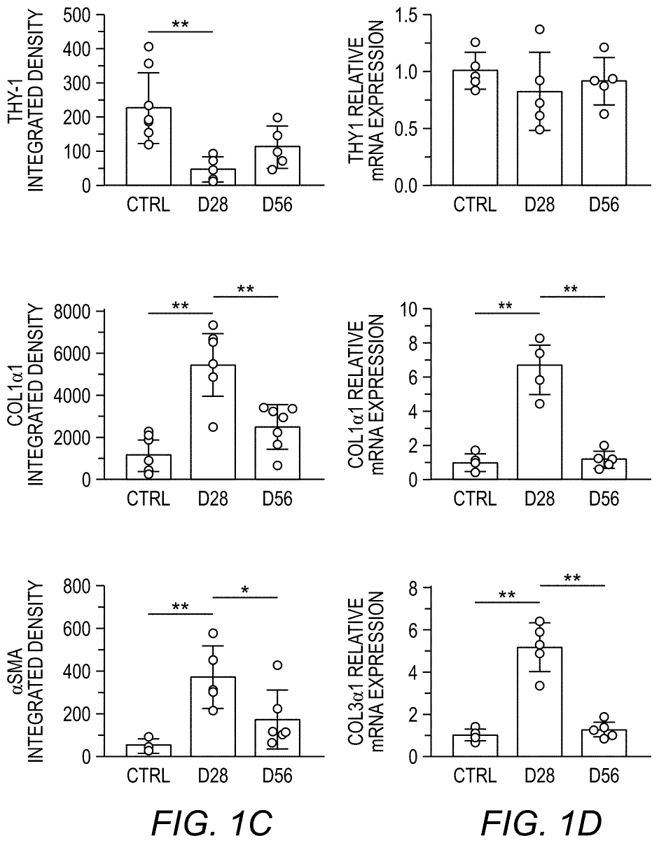

Acute Injury Induced Fibroblastic Shedding Of Thy-1 That Did Not Interrupt Lung Fibrosis: Thy-1 expression in bleomycin induced lung fibrosis was examined in transgenic reporter Col-GFP mice expressing collagen-al(I) promoter/enhancer-driven GFP (FIG. 1B). Lung fibrosis was induced by instilling bleomycin (i.t., 4 U/kg) or saline (control) and evaluated by measuring deposition of collagen and numbers of GFP⁺ fibroblasts or GFP⁺/αSMA⁺ myofibroblasts. Bleomycin-treated mice developed severe fibrosis with the accumulation of GFP⁺ cells at 28 days, which were significantly decreased following resolution of fibrosis at 56 days (FIGS. 1A, 1C, and 1D). In this model, it was found that Thy-1 immunostaining decreased mostly in GFP fibroblasts and GFP⁺/αSMA⁺ myofibroblasts 28 days following bleomycin instillation (FIGS. 1A and 1C). There were no significant changes of Thy-1 immunostaining in GFP-negative cells over time. After 8 weeks, however, Thy-1 expression was recovered in scattered fibroblasts in alveolar septae, around blood vessels, and in some areas of pleura. Examination of mRNA expression by qPCR did not demonstrate any significant time-course change of Thy-1 expression at the transcriptional level (FIG. 1D). Thus, acute injury following single dose bleomycin induces a transient loss of Thy-1 mostly at the surface protein level in activated fibroblasts likely via shedding or recycling. It seems that this transient change in Thy-1 surface expression did not affect myofibroblast disappearance and fibrosis resolution.

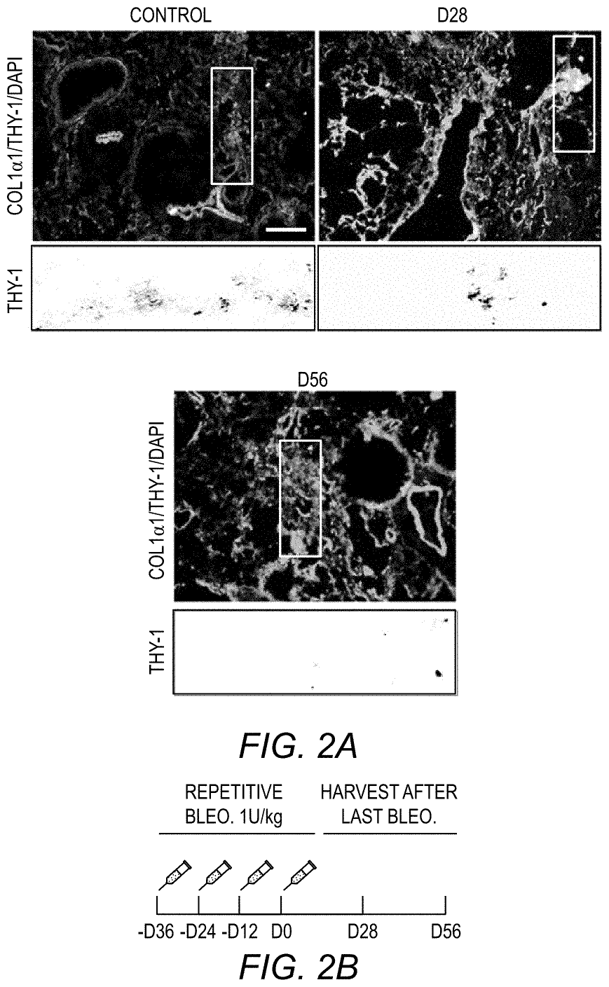

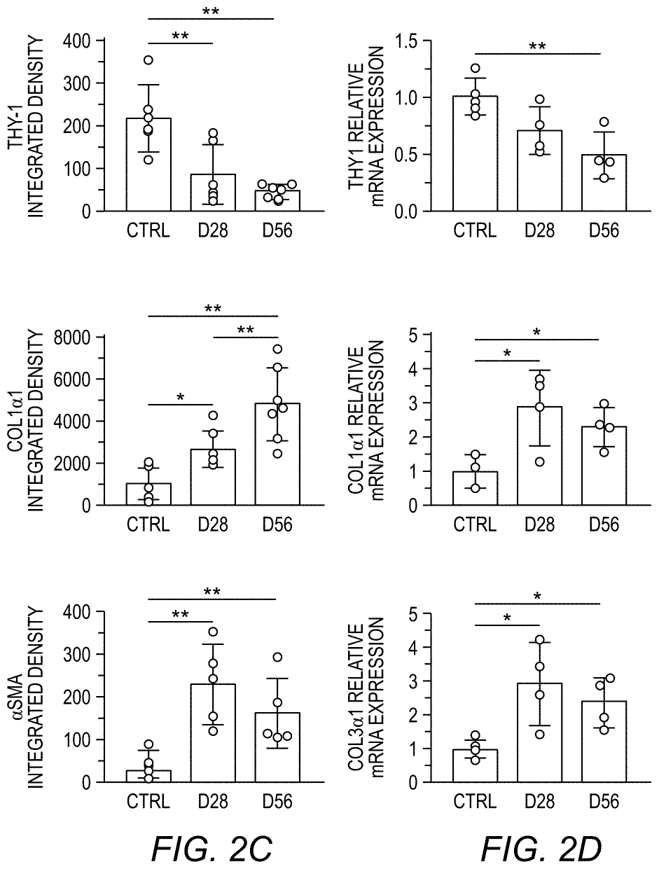

Repetitive Microinjury Resulted In Thy-1 Silencing In Myofibroblasts, Associated With Continuous Fibrotic Remodeling: Next, a repetitive lung injury model was utilized to determine whether progressive, non-resolving fibrosis is associated with sustained Thy-1 loss in fibroblasts. Col-GFP mice were subjected to 1 U/kg bleomycin or saline (control) that was instilled intratracheally every 12 days for four doses. After 28 or 56 days following the final instillation of bleomycin, lungs were evaluated by measuring Thy-1 expression, GFP⁺ and/or αSMA⁺ cells, and profibrotic gene expression (FIG. 2B). The results show that Thy-1 immunostaining disappears in GFP fibroblasts or GFP⁺/αSMA⁺ myofibroblasts at day 28 and remains low or decreases further at day 56 after final bleomycin challenge (FIGS. 2A and 2C). At both time-points, Thy1 mRNA expression in lung tissue was downregulated in a time-dependent manner (FIG. 2D). Moreover, changes in Thy-1 were accompanied by persistent accumulation of GFP⁺ and/or αSMA⁺ cells and continuous fibrotic remodeling, as measured by profibrotic genes Col1a1 and Col3a1 in lung tissue by qPCR (FIG. 2D). Together, these findings indicate that repetitive microinjury could induce Thy1 downregulation or gene silencing in addition to loss at the protein level and was associated with progressive fibrotic tissue remodeling. Thus, the emergence of persistently Thy-1(−) fibroblasts may be an important event that predisposes the lung to compromised and profibrotic repair.

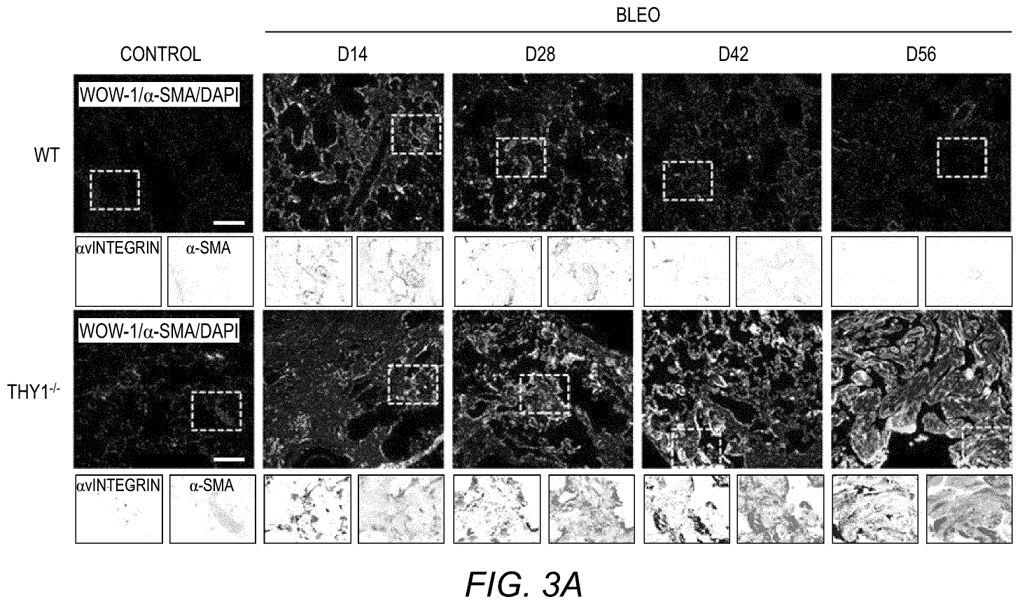

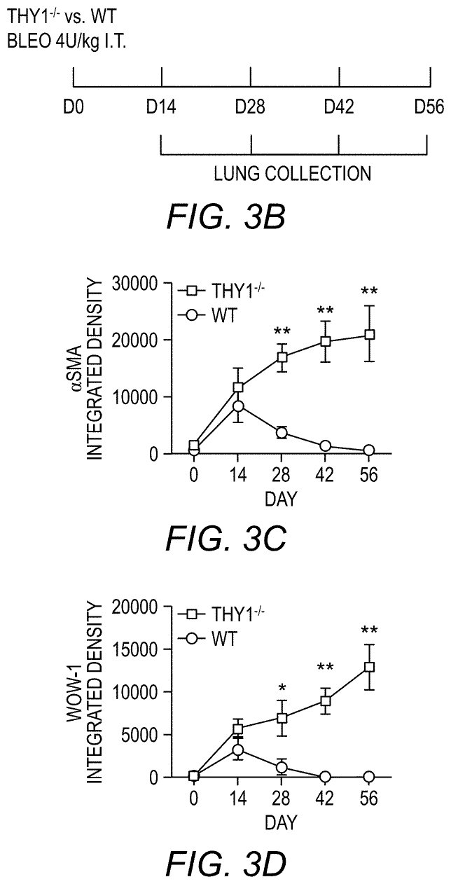

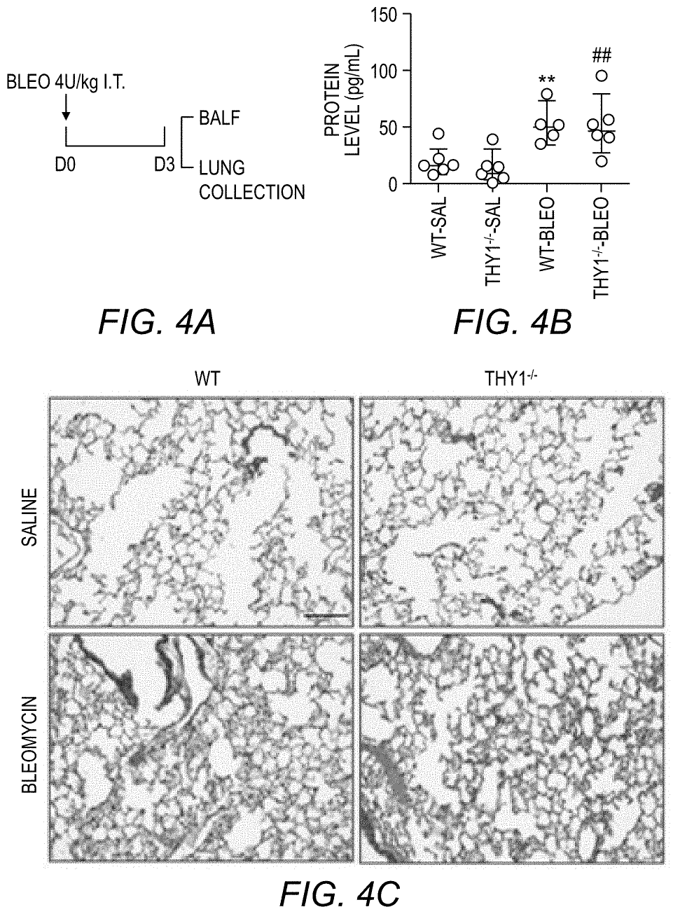

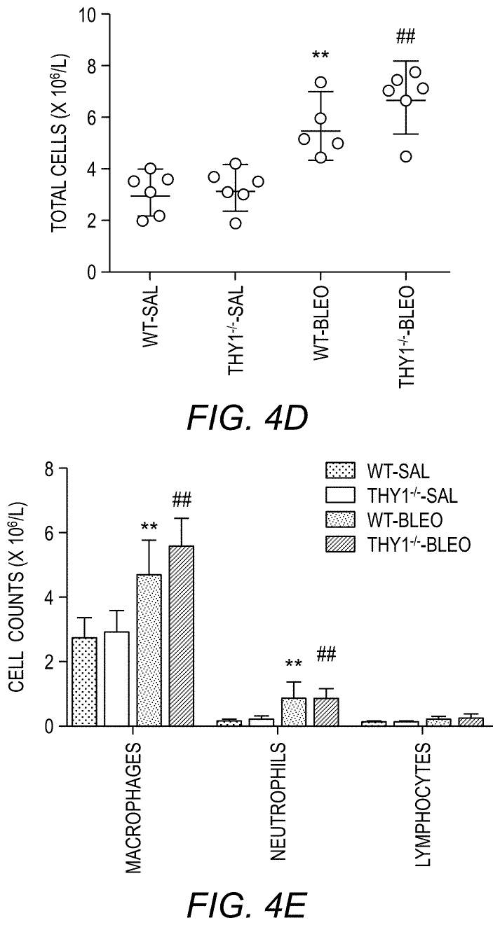

Thy-1 Loss Is Associated With Elevated Av Integrin Activity In Vivo And Progressive, Non-Resolving Fibrosis: To determine if Thy-1 loss is associated with αv integrin activation in lung fibrosis, a time course study in Thy1^{-/-} vs. wild type (WT) mice following single dose bleomycin was carried out (FIG. 3B). Thy1^{-/-} mice, unlike WT mice, fail to resolve fibrosis by 56 days post-injury. Sustained αvβ3 integrin activation in αSMA (+) myofibroblasts (indicative of active fibrosis) was observed at all time points through 56 days as determined by double IF (FIGS. 3A, 3C, and 3D). This is in contrast to WT mice that displayed a reduction in αvβ3 integrin staining after 28 days, corresponding to the initiation of resolution of bleomycin-induced fibrosis (FIGS. 3A and 3D). These findings suggest that persistently activated αv integrin in fibroblasts of mice lacking Thy-1 could result in non-resolving fibrosis. Elevated αv integrin activity at baseline in untreated Thy1^{-/-} lungs was observed, but it was not sufficient to spontaneously induce fibrosis. In the acute injury phase (3 days after bleomycin), there were no differences in histopathological alteration (H&E), lung permeability (total proteins, total counts & differentials in bronchoalveolar lavage fluid) in WT mice as compared with Thy1^{-/-} mice (FIGS. 4A-4E), suggesting that differences in early inflammation do not account for the differences in the later fibrosis. Collectively, these data demonstrated that the lung milieu in mice lacking Thy-1 promoted persistent activation/upregulation of αvβ3 integrin in fibroblasts, resulting in persistence of profibrotic myofibroblast phenotypes in vivo.

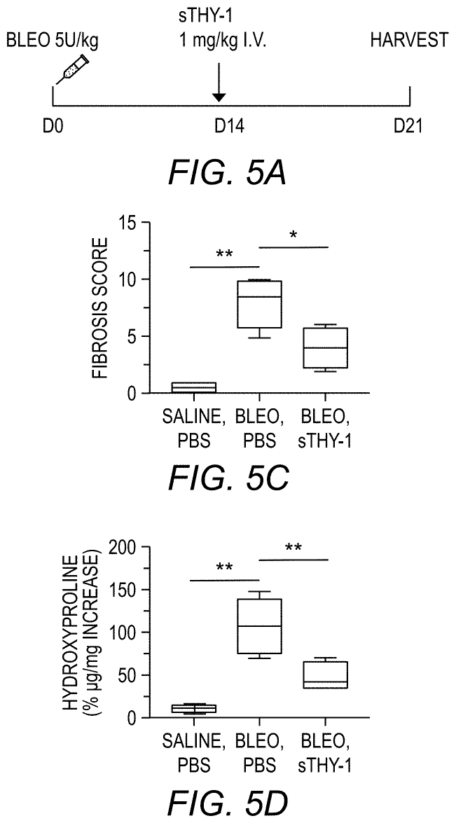

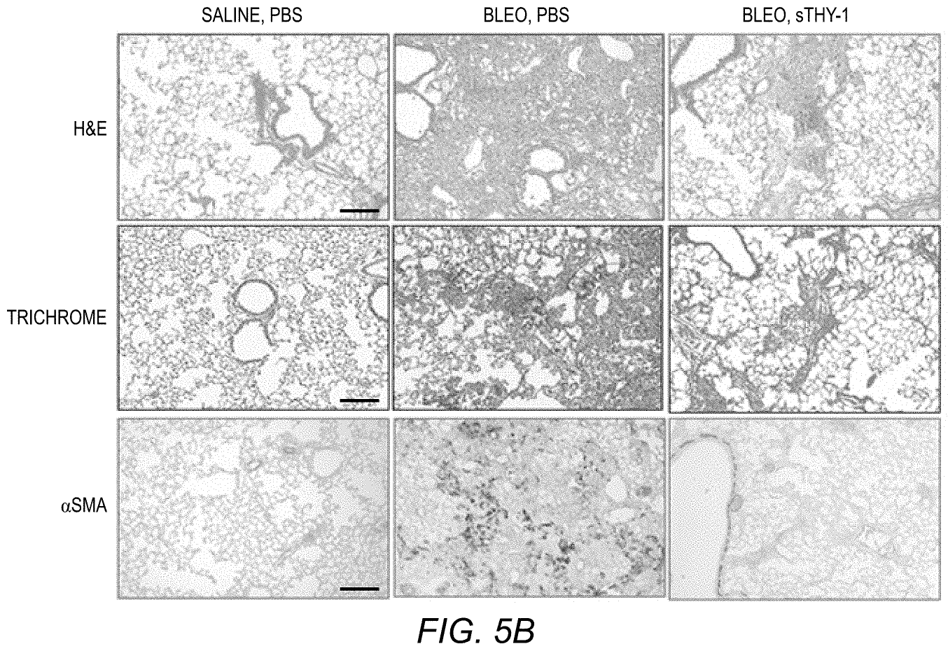

sThy-1 Promotes Resolution Of Bleomycin-Induced Fibrosis: To examine the potential therapeutic effect of a soluble form of human Thy-1 (sThy-1) in lung fibrosis, 1,000 ng/kg of sThy-1-IgG Fc or saline control was given at day 14 after single dose i.t. bleomycin instillation (i.e., after fibrosis was established) in WT mice and fibrotic parameters were assessed at day 21 (FIG. 5A). Histopathological examination shows that there was an obvious shift from a much more uniform fibrotic response to limited patchy fibrosis in mice receiving sThy-1-IgG Fc (FIG. 5B). The treatment of the mice with sThy-1-IgG Fc significantly reduced collagen deposition (FIGS. 5B and 5D) and fibrosis scores (P<0.05, FIG. 5C) when compared to bleomycin-PBS mice. IHC for αSMA (FIG. 5B) followed the same patterns as that of histopathological alterations, indicating reduction in myofibroblasts or reversal of the myofibroblast phenotype by sThy-1-IgG Fc. Administration of sThy-1-IgG Fc in saline-instilled control mice had no effect on lung histology or on the fibrotic and inflammatory endpoints. Thus, exogenous sThy-1 may have significant therapeutic potential in promoting resolution of established pulmonary fibrosis.

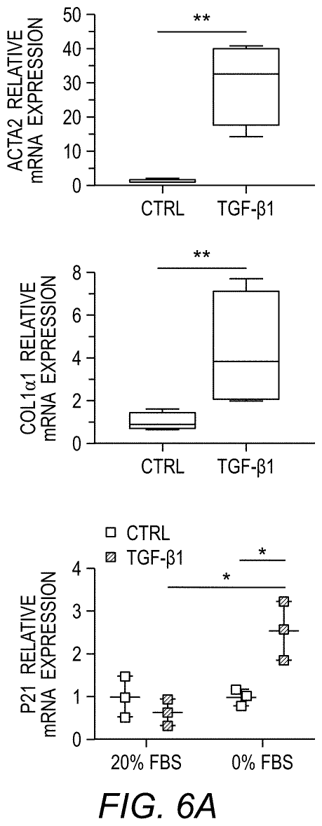

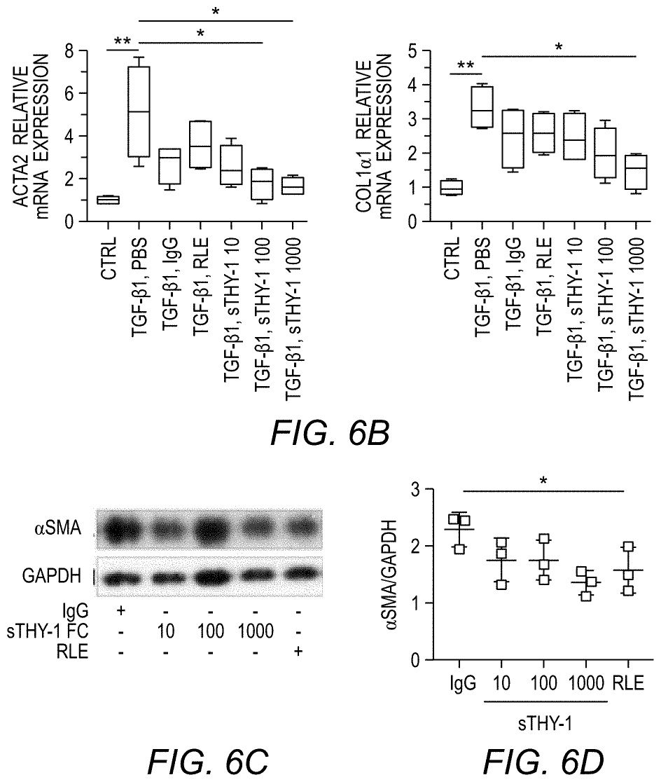

Targeting Of αv Integrin By sThy-1 Reverses Myofibroblast Differentiation In A Phenotypic In Vitro Model Of IPF Fibroblasts: Because IPF fibroblasts may be senescent and resistant to phenotype changes, the effects of sThy-1 on senescent-like myofibroblast differentiation in vitro were tested. Briefly, myofibroblast differentiation in human lung fibroblasts (CCL-210) was induced by incubation with recombinant human TGF-β1 (10 ng/ml) for 48 hours and the cells subsequently allowed to senesce by culturing for an additional 5 days in serum-free media to control proliferation. Context-dependent senescence (as evidenced by p21 expression) was induced in TGF-β1-treated and serum-fasted fibroblasts (FIG. 6A). Thus, this cellular model may be more appropriate to represent phenotypes of IPF fibroblasts. Then, these cells were treated with recombinant human sThy-1-IgG Fc (10, 100, 1,000 ng/mL), RLE-mutated Thy-1[Thy-1(RLE)-IgG Fc], IgG Fc for 48 hours. As determined by qPCR of Acta2 and Col1α1, and immunoblotting of αSMA expression, the addition of sThy-1-IgG Fc at the level of 1000 ng/mL significantly reduced myofibroblastic differentiation when compared with controls (FIGS. 6B-6D). These changes suggest that exogenous sThy-1 is effective at reversing myofibroblast differentiation, even in senescent-like cells. The modulatory effects by sThy-1-IgG Fc, but not Thy-1(RLE)-IgG Fc, indicated that the RGD-like integrin-binding motif in Thy-1 is required.

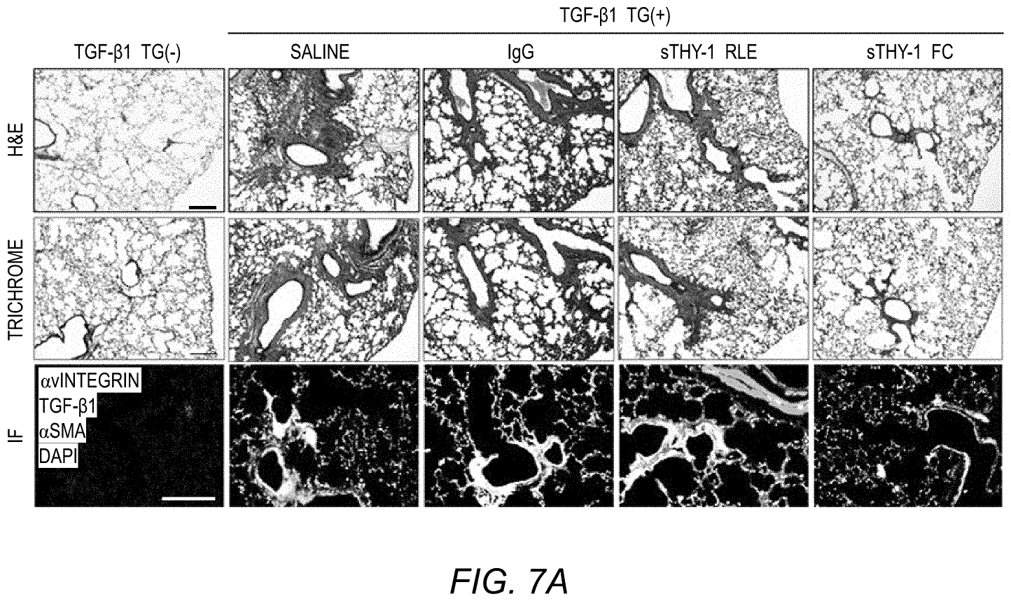

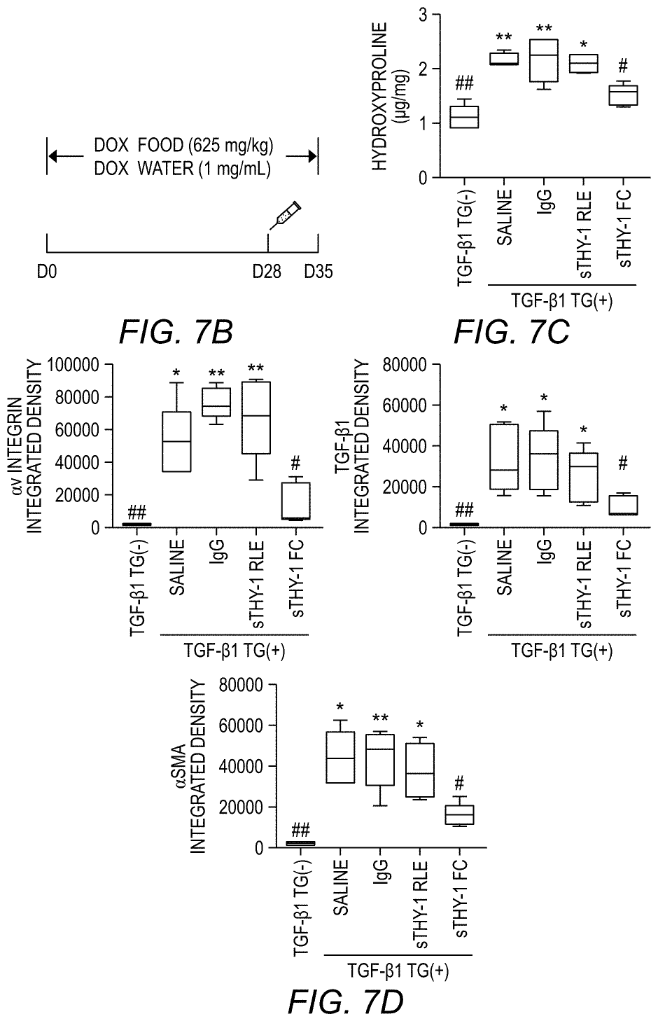

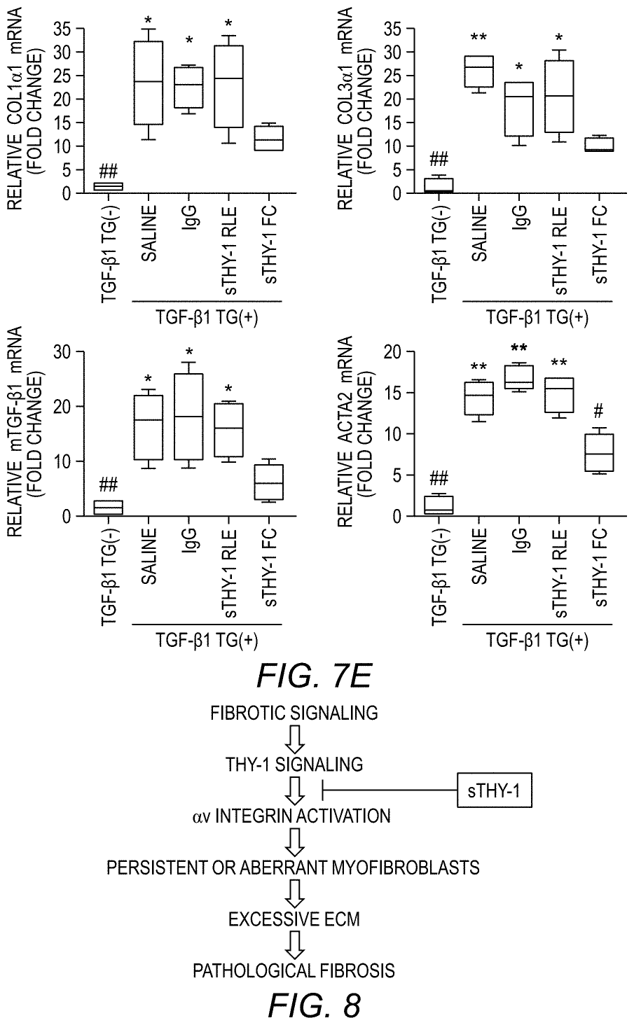

sThy-1 Reverses Established TGF-β1-induced Lung Fibrosis In Mice: To determine the therapeutic potential of sThy-1 in an alternative, TGF-β1-driven genetic model of fibrosis, the effect of human sThy-1 Fc on Dox-induced expression of active human TGF-β1 driving lung fibrosis was investigated in CC10-rtTA-tTS-Tgfb1 Tg(+) mice (FIG. 7B). 28 days after Dox induction, CC10-rtTA-tTS-Tgfb1 Tg(+) mice showed apparent airway and alveolar parenchymal fibrotic response, characterized by alveolar septal thickening and areas of septal rupture as determined by histopathological analyses (H&E and Trichrome stains, FIG. 7A). Treatment of these mice at day 28 with a 1,000 ng/kg i.v. of sThy-1-IgG Fc, but not sThy-1(RLE) or IgG, showed resolution of airway and alveolar parenchymal fibrotic response (FIG. 7A). Administration of sThy-1 Fc resulted in a significant reduction in the level of hydroxyproline (FIG. 7C), expression of αv integrin, murine TGF-β1, and αSMA measured by immunofluorescence (FIG. 7D), and reduction in expression of profibrotic genes (Col1a1, Col3a1, Tgfb1 and Acta2) as measured by qPCR (FIG. 7E). These changes indicated that the exogenous sThy-1 polypeptide is effective at reversing myofibroblast differentiation and established fibrosis in vivo and is associated with decreased αv integrin activation. The therapeutic effect of sThy-1-IgG Fc, but not sThy-1(RLE)-IgG Fc, indicated that the Thy-1 RLD (RGD-like motif) is required. Taken together, these data demonstrate that soluble human Thy-1 reverses lung fibrosis via trans regulation of αv integrin activity in Dox induced CC10-rtTA-tTS-Tgfb1 Tg(+) mice, underscoring its therapeutic potential in resolution of established pulmonary fibrosis in two distinct models of fibrosis.

In these studies, a critical homeostatic role of a primary Thy-1-αv integrin interaction upstream of canonical ECM-integrin ligation in vivo was demonstrated, disruption of which is associated with progressive fibrogenesis following lung injury. In a severe acute lung injury, such as in the single dose bleomycin model, loss of Thy-1 is incomplete and transient, and recovery of Thy-1 expression occurs during fibrosis resolution. However, repetitive administration of lower doses of bleomycin induced progressive, non-resolving lung fibrosis associated with sustained transcriptional silencing of Thy1 expression in myofibroblasts. Although transient loss of Thy-1 is related to fibroblast activation, silencing of Thy1 may initiate a more permanent shift toward a Thy-1-negative, profibrotic, apoptosis-resistant myofibroblast phenotype. Epigenetic silencing of Thy1 expression in lesional fibroblasts in fibroblastic foci of pulmonary fibrosis, and demonstrated reversal of myofibroblastic differentiation associated with restored Thy1 expression following epigenetic modifiers in vitro has been previously demonstrated. This paradigm has been demonstrated in vivo in the context of aging and TGF-β1, as well as in the context of sustained TLR4 activation.

Additionally, sustained absence of Thy-1 in the context of lung injury (such as in Thy1^{-/-} mice following bleomycin) is associated with progressive, non-resolving fibrosis, and as shown here, sustained activation of αv integrin. The activated αv integrin due to Thy-1 loss facilitates myofibroblast differentiation, likely via multiple downstream pathways. The data showed sThy-1 could reverse TGF-β1-induced myofibroblast differentiation in vitro, likely via trans-regulation of αv integrins, even in the context of induced senescence. Furthermore, treatment of Dox-induced CC10-rtTA-tTS-Tgfb1 mice and bleomycin induced mice with sThy-1 demonstrated an antifibrotic efficacy. Collectively, these findings suggest that targeting of αv integrin by sThy-1 molecules could be a novel strategy to treat lung fibrosis, possibly halting the progression or speeding the resolution of lung fibrosis.

In vitro studies indicated the potential of sThy-1, via integrin engagement, to reverse the experimental induction of the myofibroblastic phenotype, but its antifibrotic efficacy in vivo, where fibrosis is a more complex pathophysiologic process, had not been previously determined. To this end, the effects of exogenously administered sThy-1 on established lung fibrosis (14 days post bleomycin) was examined. The data clearly support the therapeutic effect of sThy-1 in promoting resolution of fibrosis, especially indicating reduction in myofibroblasts (or reversal of the myofibroblast phenotype) by sThy-1. The beneficial effect of a single treatment with sThy-1 suggests a “reset” of the fibrogenic program. Because single-dose bleomycin-induced fibrosis is self-resolving, sThy-1 in this context may act to facilitate or accelerate endogenous programs for resolution. In order to test the therapeutic potential of sThy-1 in a more progressive model, CC10-rtTA-tTS-Tgfb1 mice were selected, in which transgene induction with Dox results in expression of the active form of human TGFβ1, which initiates a self-sustaining fibrogenic milieu. Intervention occurred at a later time point (day 28) when fibrotic remodeling is well established. Remarkably, a single administration of sThy-1 promoted significant resolution of fibrosis over the ensuing 7 days with improvement in multiple histologic and biochemical measures of fibrosis, including expression of endogenous TGF-β1. The effect is dependent on the Thy-1 RLD sequence, which is accompanied by decreased activation of αv integrin, suggesting engagement of an important homeostatic axis. The cellular and molecular mechanisms of this effect are likely to be complex, but could involve restoration of mechanosensitive signaling, myofibroblast apoptosis, and interruption of latent TGF-β1 activation. The modulation of fibroblast senescence may possibly be involved in the Thy-1−/integrin/TGF-β1 pathway. It is unknown whether trans-signaling from exogenous sThy-1 alters epigenetic mechanisms regulating to Thy1 expression.

This study has important implications for therapeutic development. Most antifibrotic agents limit ongoing fibrogenesis, but do not promote resolution of existing fibrosis. sThy-1 seems to have the latter activity, and may be useful in a disease such as IPF, which displays temporal heterogeneity with areas of active fibrogenesis alternating with areas of more established fibrosis. The fibrogenic phase of acute lung injury, which has the capacity to resolve but also has an extremely high mortality rate and is subject to epigenetic alterations which promote progression, may benefit from the pro-resolution “reset” activity seen in these studies. There is significant enthusiasm for cell-based therapies for acute lung injury and fibrosis, many of which use mesenchymal stem cells (which are strongly Thy-1⁺) or their products to restore a homeostatic regenerative milieu. Experts advise tempering such enthusiasm with appropriate caution, as cell-based regenerative therapies are complex and may have unanticipated long-term consequences. sThy-1 may be able to mediate some of the beneficial effects of cell-based therapies with fewer concerns, especially if it can be administered in a single administration, or a series of intermittent infusions (FIG. 8).

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. (canceled)

2. A method of treating, inhibiting, and/or reversing tissue fibrosis in a subject in need thereof, comprising delivering to the subject a therapeutically effective amount of a soluble Thy-1 polypeptide or a functional fragment thereof or a fusion protein comprising a soluble Thy-1 polypeptide or a functional fragment thereof, thereby treating, inhibiting, and/or reversing tissue fibrosis in the subject.

3. The method of claim 2, wherein the fibrosis is self-resolving fibrosis.

4. The method of claim 2, wherein the fibrosis is non-resolving fibrosis.

5. The method of claim 2, wherein the fibrosis is lung fibrosis.

6. The method of claim 5, wherein the lung fibrosis is idiopathic lung fibrosis.

7. The method of claim 2, wherein the fibrosis is kidney, liver, or heart fibrosis.

8. A method of inhibiting alveolar septal thickening in a subject in need thereof, comprising delivering to the subject a therapeutically effective amount of a soluble Thy-1 polypeptide or a functional fragment thereof or a fusion protein comprising a soluble Thy-1 polypeptide or a functional fragment thereof, thereby inhibiting alveolar septal thickening in the subject.

9. A method of treating or preventing a disorder responsive to inhibition or reversal of tissue fibrosis in a subject in need thereof, comprising delivering to the subject a therapeutically or prophylactically effective amount of a soluble Thy-1 polypeptide or a functional fragment thereof or a fusion protein comprising a soluble Thy-1 polypeptide or a functional fragment thereof, thereby treating or preventing the disorder in the subject.

10. The method of claim 2, wherein the soluble Thy-1 polypeptide or a functional fragment thereof or the fusion protein comprising a soluble Thy-1 polypeptide or a functional fragment thereof is delivered by inhalation.

11. The method of claim 2, wherein the soluble Thy-1 polypeptide or a functional fragment thereof or the fusion protein comprising a soluble Thy-1 polypeptide or a functional fragment thereof is delivered systemically, such as intravenously.

12. The method of claim 2, wherein the fusion protein comprising a soluble Thy-1 polypeptide or a functional fragment thereof is a fusion with an immunoglobulin Fc region.

13. The method of claim 2, wherein the fusion protein comprising a soluble Thy-1 polypeptide or a functional fragment thereof does not comprise an immunoglobulin Fc region.

14. The method of claim 2, wherein the functional fragment of Thy-1 is a deletion of a glycosylphosphatidyl inositol attachment signal.

15. The method of claim 2, wherein the Thy-1 is human Thy-1.

16. The method of claim 2, wherein the subject is a human.

17-34. (canceled)

35. The method of claim 11, wherein the soluble Thy-1 polypeptide or a functional fragment thereof or the fusion protein comprising a soluble Thy-1 polypeptide or a functional fragment thereof is delivered intravenously.


SEQUENCE LISTING (15 sequences)

SEQ ID NO: 1 - protein, 161 aa, synthetic construct
MNLAISIALLLTVLQVSRGQKVTSLTACLVDQSLRLDCRHENTSSSPIQYEFSLTRETKKHVLFGTVGVPEHTYRSRTNFTSKYHMKVLYLSAFTSKDEGTYTCALHHSGHSPPISSQNVTVLRDKLVKCEGISLLAQNTSWLLLLLLSLSLLQATDFMSL

SEQ ID NO: 2 - DNA, 20 bp, synthetic construct
  source 1..20: mol_type = other DNA
gggcgactacttttgtgagc

SEQ ID NO: 3 - DNA, 20 bp, synthetic construct
  source 1..20: mol_type = other DNA
tctgaaccagcaggcttatg

SEQ ID NO: 4 - DNA, 21 bp, synthetic construct
  source 1..21: mol_type = other DNA
acatgttcagctttgtggacc

SEQ ID NO: 5 - DNA, 20 bp, synthetic construct
  source 1..20: mol_type = other DNA
taggccattgtgtatgcagc

SEQ ID NO: 6 - DNA, 22 bp, synthetic construct
  source 1..22: mol_type = other DNA
attgctgggatcactggagcac

SEQ ID NO: 7 - DNA, 24 bp, synthetic construct
  source 1..24: mol_type = other DNA
cctggtttcccactttcacccttg

SEQ ID NO: 8 - DNA, 20 bp, synthetic construct
  source 1..20: mol_type = other DNA
actgggacgacatggaaaag

SEQ ID NO: 9 - DNA, 20 bp, synthetic construct
  source 1..20: mol_type = other DNA
gttcagtggtgcctctgtca

SEQ ID NO: 10 - DNA, 20 bp, synthetic construct
  source 1..20: mol_type = other DNA
ggagagccctggataccaac

SEQ ID NO: 11 - DNA, 20 bp, synthetic construct
  source 1..20: mol_type = other DNA
caacccaggtccttcctaaa

SEQ ID NO: 12 - DNA, 22 bp, synthetic construct
  source 1..22: mol_type = other DNA
ctaaggccaaccgtgaaaagat

SEQ ID NO: 13 - DNA, 20 bp, synthetic construct
  source 1..20: mol_type = other DNA
cacagcctggatggctacgt

SEQ ID NO: 14 - DNA, 20 bp, synthetic construct
  source 1..20: mol_type = other DNA
atcgagacatgtgatcaagc

SEQ ID NO: 15 - DNA, 20 bp, synthetic construct
  source 1..20: mol_type = other DNA
gcgtgcataaattgtatagc